(19) United States
(12) Patent Application Publication
(10) Pub. No.: US 2022/0257296 A1
(43) Pub. Date: Aug. 18, 2022

(54) ORBITAL FLOOR TEMPLATE

(71) Applicant: Poriferous, LLC, Newnan, GA (US)

(72) Inventor: Aaron NOBLE, Newnan, GA (US)

(73) Assignee: Poriferous, LLC, Newnan, GA (US)

(21) Appl. No.: 17/597,859

(22) PCT Filed: Mar. 19, 2020
(86) PCT No.: PCT/US2020/023604

§ 371 Date: Jan. 26, 2022

Related U.S. Application Data
(60) Provisional application No. 62/820,320, filed on Mar. 19, 2019.

(51) Int. Cl. A61B 17/88

(52) U.S. Cl. CPC A61B 17/8863

(57) ABSTRACT

An orbital floor template packaged along with an implant. The two components are provided together in a single sterile package. This allows the surgeon to pull a single package and have the implant and a pre-sized template available during surgery. This avoids the need to pull a second implant from the operating room materials shelf for sizing purposes only. The template is used during surgery and prevents the need to use a separate, new, sterile, separately-packaged actual implant for sizing purposes.

### BACKGROUND

During orbital or other craniofacial implant surgery, a surgeon may require a template in order to appropriately shape a final implant. In current practice, the surgeon typically pulls two final implants from stock. One will be the actual implant that will be positioned and remain implanted in the patient, and the second implant is used as a sizing template, even though this implant is of quality that it could also be positioned and remain in the patient. This is ultimately wasteful; a separate implant is used during surgery, but at implant cannot be charged for (either through insurance or to the patient).

### FIELD OF THE DISCLOSURE

The present disclosure relates generally to an orbital floor template. The template is used during surgery and prevents the need to use a separate, new, sterile, separately-packaged actual implant for sizing purposes.

### BRIEF DESCRIPTION OF THE DRAWINGS

FIG. 1 shows a perspective view of a patient's orbital floor with a template in use to size the implant.

FIG. 2 shows a schematic of a template and an implant packaged together in sterile packaging.

### SUMMARY

The present disclosure thus provides an implant that is packaged along with an orbital floor template. The two components are provided together in a single sterile package. This allows the surgeon to pull a single package and have the implant and a pre-sized template available during surgery. This avoids the need to pull a second implant from the operating room materials shelf for sizing purposes only.

### DETAILED DESCRIPTION

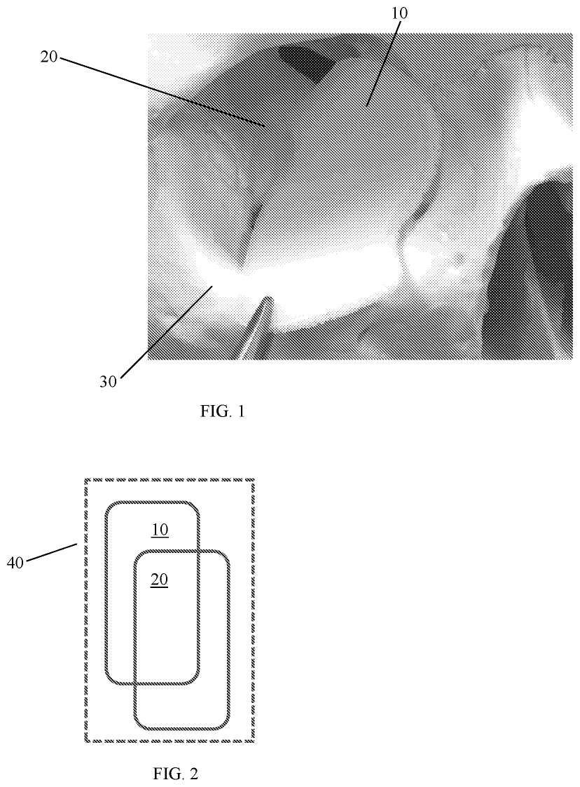

The accompanying figures illustrate the process for which the template may be used. FIG. 1 is a close-up illustrating an implant 10 (shown as porous material) being sized by use of the disclosed template 20 (shown as a nonporous, thinner component, overlaid over the implant).

In some examples, the template 20 is a non-porous material. The template 20 is made of a material that can be bent and retain its shape. It is generally sized to correspond to the orbital floor implant 10. Various sizes of the template 20 may be provided along with variously sized implants 10. In some examples, the templates 20 are closely sized to match the implants 10. The template 20 generally corresponds to the orbital floor 30 shape. For example, it may be provided with a curvature similar to a curvature of the implant. The template may be provided of the same biocompatible materials of which the implant is made. Specifically, the template can be made from the same biocompatible HDPE (high density polyethylene) as the implant material. Rather than providing a template of a different material, this embodiment uses the same polymer material for the template that is used for the implant. This reduces risk of a non-implantable material being adversely introduced into the patient. For example, other templates have been made of silicone or other non-implant grade material. By contrast, using a polyethylene material for both the template and the implant can reduce risks or other complications. The template could be used as an implant itself as it is of the same implant grade material.

In one example, implants are typically about 0.85-1.0 mm thick. The disclosed template 20 be about 0.20 to 0.5 mm thick. In a specific example, disclosed template 20 may be about 0.4 mm thick. It is generally envisioned that the template 20 can be thinner than the implant 10, but this is not required. The template 20 is preferably designed so that it is able to be cut via a scalpel or surgical scissors. This can assist the surgeon during shaping and placement of the procedure.

A primary benefit of the disclosed template 20 is that it may be included in a sterile pouch or packaging 40 with the implant 10, as shown by FIG. 2. For example, if an implant is $100, it may be provided with a template, preventing the use of two implants at $200. This can save costs for hospitals, patients, and insurance companies, and came save time for surgeons. The template could be included at a nominal charge or not be charged at all.

It should be understood that various different features described herein may be used interchangeably with various embodiments. For example, if one feature is described with respect to particular example, it is understood that that same feature may be used with other examples as well.

Changes and modifications, additions and deletions may be made to the structures and methods recited above and shown in the drawings without departing from the scope or spirit of the disclosure or the following claims.

1. An orbital floor implant and template kit, comprising:
an orbital floor implant, and
a template shaped to correspond to the orbital floor implant.

2. The kit of claim 1, wherein the template and the orbital floor implant are made of the same polymeric material.

3. The kit of claim 1, wherein the template and the orbital floor template comprise high density polyethylene.

4. The kit of claim 1, wherein the template is made of nonporous material.

5. The kit of claim 1, wherein the implant is made of porous material.

6. The kit of claim 1, wherein the template can be bent and retain the shape to which is has been bent.

7. The kit of claim 1, wherein the template can be cut with scissors or a scalpel.

8. The kit of claim 1, wherein the template is about 0.4 mm thick.

9. The kit of claim 1, wherein the implant and the template are provided together in a sterile package.

10. The kit of claim 1, wherein the template is made of a biocompatible material.
